(19) United States
(12) Patent Application Publication
(10) Pub. No.: US 2012/0265121 A1
(43) Pub. Date: Oct. 18, 2012

(54) PHTHALOCYANINE-BASED ANTIFUNGAL AGENTS

(75) Inventors: Elma D. Baron, Concord, OH (US); Kevin D. Cooper, Moreland Hills, OH (US); Minh Lam, Highland Heights, OH (US); Pranab Mukherjee, Beachwood, OH (US); Mahmoud A. Ghannoum, Shaker Heights, OH (US); Nancy L. Oleinick, University Heights, OH (US)

(73) Assignee: CASE WESTERN RESERVE UNIVERSITY, Cleveland, OH (US)

(21) Appl. No.: 13/423,563

(22) Filed: Mar. 19, 2012

Related U.S. Application Data
(60) Provisional application No. 61/454,168, filed on Mar. 18, 2011.

(51) Int. Cl. A61M 37/00

(52) U.S. Cl. 604/20

(57) ABSTRACT

A method is described for the photodynamic treatment of a fungal infection in a subject by administering a therapeutically effective amount of a phthalocyanine compound or a pharmaceutically acceptable salt thereof to the subject and activating the phthalocyanine compound with light. The method is useful for treating various dermatophyte infections such as onychomycosis, and in particular fungal infection by Candida and Trichophyton.

### RELATED APPLICATION DATA

This application claims the benefit of U.S. Provisional Application Ser. No. 61/454,168, filed Mar. 18, 2011, which is incorporated by reference herein.

### GOVERNMENT FUNDING

This work was supported, at least in part, by the Skin Diseases Research Center Grant Number 5P30AR39750 from the National Institute of Health. The United States government has certain rights in this invention.

### BACKGROUND OF THE INVENTION

Photodynamic therapy (PDT) involves the interaction of light, a photosensitizing agent, and molecular oxygen to induce a biologic response. It is currently utilized in eradicating cancers of the skin, gastrointestinal tract, lungs, and urinary bladder. The silicon phthalocyanine Pc 4 is a second generation photosensitizer developed at Case Western Reserve University. Upon activation by 675-nm wavelength red light, the excited Pc 4 molecule interacts with oxygen present in the tissue and forms reactive oxygen species, which then act on proteins or lipids within their immediate vicinity. Pc 4 also possesses a higher molar extinction coefficient at longer wavelengths than currently approved photosensitizers (e.g. Photofrin®), translating into more avid absorption of light. In addition, pharmacokinetic data in mice indicate that when delivered systemically, Pc 4 is much more rapidly cleared than Photofrin®, thereby minimizing the possibility of prolonged generalized photosensitivity. Oleinick et al., Photochem Photobiol 57:242-247 (1993).

Infections due to Candida and other fungi continue to represent a significant health burden. Some cases are highly resistant to traditional antifungals such as fluconazole and amphotericin B. Even with antifungal therapy, mortality of patients with invasive candidiasis can still be as high as 40 percent. Candidiasis is usually associated with indwelling medical. devices (e.g. dental implants, catheters, heart valves, vascular bypass grafts, ocular lenses, artificial joints and central nervous system shunts), which can act as substrates for biofilm growth. Often, removal of the catheters/devices is warranted to treat the infection. Biofilm formation is also critical in the development of denture stomatitis, a superficial form of candidiasis that affects 65% of edentulous individuals. Calderone et al., Microbiol. Rev; 55:1-20 (1991). Despite the use of antifungal drugs to treat denture stomatitis, infection is often re-established soon after treatment. These observations emphasize the clinical significance of fungal biofilm formation and the inability of commonly used antifungals (e.g. fluconazole, amphotericin B) to cure such diseases.

In vitro studies have demonstrated that Candida species are susceptible to PDT using Photofrin® (Bliss et al., Antimicrob. Agents Chemother.; 48;2000-06 (2004)) or the porphyrin precursor 5-aminolaevulinic acid (ALA). Monfrecola et al., Photochem. Photobiol.; 3:419-422 (2004). Other photosensitizers that are not yet FDA approved have also shown the ability to inhibit Candida species. Chabrier-Rosello et al., Photochem Photobiol 84:1141-1148 (2008); and de Souza et al., J Photochem Photobiol B 83:34-38 (2006).

However, infections due to Candida and other fungi continue to represent a significant health burden. Some cases are highly resistant to traditional antifungals such as fluconazole and amphotericin B. Even with antifungal therapy, mortality of patients with invasive candidiasis can still be as high as 40 percent. Candidiasis is usually associated with indwelling medical. devices (e.g. dental implants, catheters, heart valves, vascular bypass grafts, ocular lenses, artificial joints and central nervous system shunts), which can act as substrates for biofilm growth. Often, removal of the catheters/devices is warranted to treat the infection. Biofilm formation is also critical in the development of denture stomatitis, a superficial form of candidiasis that affects 65% of edentulous individuals. Despite the use of antifungal drugs to treat denture stomatitis, infection is often re-established soon after treatment. MacEntee, M. I. J. Oral Rehabil. 12:195-207 (1985). These observations emphasize the clinical significance of fungal biofilm formation and the inability of commonly used antifungals (e.g. fluconazole, amphotericin B) to cure such diseases.

Fungal infection of the nail or onychomycosis is the most common nail disease in adulthood, with an incidence as high as 13% in North America. It accounts for half of all nail diseases and affects as much as 90% of elderly individuals. Treatment is limited because topical antifungals are largely ineffective and systemic agents have serious adverse effects such as liver damage. Whereas the most common etiologic agent for onychomycosis is Trichophyton rubrum, this disease can also be caused by Candida albicans, especially in immunocompromised individuals. Treatment of onychomycosis is challenging because the infection is embedded within the nail and is difficult to reach; full removal of symptoms is slow and may take a year or more. A need therefore remains for additional methods for treating fungal infection, and in particular methods that are effective for treating onychomycosis.

### SUMMARY OF THE INVENTION

In inventors have surprisingly demonstrated that phthalocyanine compounds are selectively uptaken by fungal cells, and that the fungal cells including phthalocyanine compounds can be destroyed by photodynamic therapy. Accordingly, in one aspect, the invention provides a method of photodynamic treatment of a fungal infection in a subject in need thereof. The method includes administering a therapeutically effective amount of a phthalocyanine compound or a pharmaceutically acceptable salt thereof to the subject and activating the phthalocyanine compound with light. Various additional phthalocyanine compounds, such as those including an axial ligand bearing an amine, are used in additional embodiments. In a preferred embodiment, the phthalocyanine compound is Pc4.

When photodynamic treatment is conducted using Pc4, embodiments of the invention can provide light having a wavelength of from about 660 nm to about 680 nm. In additional embodiments, photodynamic treatment includes administering from about 0.5 to about 2.0 J/cm² of light.

Photodynamic therapy using phthalocyanine compounds can be used to treat various different types of fungal infection. In some embodiments, the fungal infection is caused by yeast of the genus Candida. In a further embodiment, the fungal infection is caused by Candida albicans. In other embodiments, the fungal infection is caused by Trichophyton rubrum. In additional embodiments, the fungal infection is onychomycosis, such as Candidal onychomycosis.

In many embodiments, the phthalocyanine compound is administered topically. However, in other embodiments, the phthalocyanine compound can delivered through alternate routes and with alternate formulations, such as those suitable for systemic administration. In some embodiments, the phthalocyanine compound administered in a pharmaceutical carrier at a concentration from about 0.05 mg/ml to about 0.1 mg/ml.

### BRIEF DESCRIPTION OF THE FIGURES

The present invention may be more readily understood by reference to the following figures wherein:

FIG. 1 provides a bar graph showing that Pc 4 impairs fungal metabolic activity in C. albicans. Values are shown as percent viability, normalized to the viability of the untreated controls and are an average of three independent experiments. A. 2 J/cm² only; B. 200 nM Pc 4+0.2 J/cm²; C. 200 nM Pc 4+0.5 J/cm²; D. 200 nM Pc 4+1.0 J/cm²; E. 200 nM Pc 4+2.0 J/cm²; F. 1.0 μM Thapsigargin; G. 30% ethanol.

FIG. 2 shows that Pc 4-PDT is lethal to C. albicans as measured by CFUs. FIG. 2(a) provides a bar graph showing the results from treatment of Candida cell cultures at equal concentrations per hemocytometer counts with 0.1-1.0 μM Pc 4 for at least 2 hr before being irradiated with 2.0 J/cm² of 670-675 nm light and plated on Sabouraud dextrose agar for 24 hr incubation at 37° C. The percentage of surviving cells from the PDT-treated cultures was normalized against that of cultures exposed to Pc 4 or light alone (plating efficiencies of control cultures >50%). Values represent the mean from at least three independent experiments. Error bars indicate S.E. For all Pc 4-PDT-treated cultures vs. untreated cells, either light alone or Pc 4 alone, p<0.005. FIG. 2(b) provides an image; for a qualitative assessment of C. albicans survivability post Pc 4-PDT treatment, all yeast samples with varying concentrations of Pc 4 plus controls were plated on one 100-mm petri dish. All cell suspensions were prepared as in (a). One 100-mm petri dish with Sabouraud dextrose agar was divided into eight equal wedges. After Pc 4-PDT treatment, one drop of each cell suspension as well as light and dark controls were placed on each wedge and incubated for 24 hours at 37° C.

FIG. 3 shows that Pc 4-PDT leads to the loss of metabolic activity in C. albicans as measured by XTT assay and FUN-1 fluorescence. FIG. 3(a) Candida cell cultures at equal concentrations per hemocytometer counts were treated with 0.1-1.0 μM Pc 4 for at least 2 hr and then were irradiated with 2.0 J/cm² of 670-675 nm light. One hour following photo-irradiation of Pc 4-loaded cells, cultures were then assayed for XTT reduction. Values represent mean±S.E. from at least three independent experiments for all Pc 4-PDT-treated cultures, p<0.005 versus light alone control. FIG. 3(b) Candida cells cultures were treated with (lower panel) or without (upper panel) 1.0 μM Pc 4 for at least 2 hr and then both were irradiated with 2.0 J/cm² of 670-675 nm light. One hour following photo-irradiation of Pc 4-loaded cells, cultures were loaded with FUN-1 probe which was detected by confocal microscopy. Scale bar, 10 μm.

FIG. 4 shows the quantification of apoptosis as characterized by phosphatidylserine exposure in C. albicans: Candida cell cultures were treated with either 1.0 μM Pc 4 for at least 2 hr and then were irradiated with 2.0 J/cm² of 670-675 nm of light or 20 mM H₂O₂. Cells were then incubated for 4 hr before staining with FITC-labeled Annexin V. FIG. 4(a) provides confocal images of FITC-labeling of cells that had been treated with H₂O₂ or Pc 4-PDT. Scale bar, 20 μm. FIG. 4(b) provides a bar graph showing the results. For each experiment over 50-100 protoplasts cells were counted per sample. The experiments were performed as seen in (a) together with 60 μM of acetic acid, untreated cells and those given Pc 4 alone. Cells were counted by eye using a Zeiss Axiovert™ S100 inverted epifluorescence microscope. Values represent the mean±S.E. from at least three independent experiments. For all cultures treated with H₂O₂ or acetic acid or Pc 4-PDT, p<0.05 vs. the combined controls (untreated cells and those given either Pc 4 alone or light alone).

FIG. 5 shows (a) the growth of the microconidia isolated from terbinafine sensitive T. rubrum (23013) was inhibited by Pc 4-PDT in a dose response fashion, (b) the growth of the microconidia isolated from terbinafine resistant T. rubrum (MRL66) was inhibited by Pc 4-PDT in a dose response fashion, (c) the metabolic activity of both Terbinafine sensitive (23013) and resistant (MRL666) strain attenuates following Pc 4-PDT as demonstrated by XTT assay.

FIG. 6 shows that Pc 4-PDT induces cell death in T. rubrum (23012 and MRL 666) as measured by CFUs. Cells were treated with 0-2 μM of Pc 4 for at least 2 hours before being photoirradiated with 2.0 J/cm². Light alone did not induce cell death or affect cell proliferation.

### DETAILED DESCRIPTION OF THE INVENTION

A method of photodynamic treatment a fungal infection in a subject in need thereof is described herein. The method includes administering a therapeutically effective amount of a phthalocyanine compound or a pharmaceutically acceptable salt thereof to the subject and activating the phthalocyanine compound with light.

Embodiments of the invention may use phthalocyanine compounds as photosensitizers. A wide variety of phthalocyanine compounds have been developed, having a variety of absorption wavelengths, solubilities, and other characteristics. See for example U.S. patent application Ser. No. 10/599,433, entitled “Topical Delivery of Phthalocyanines” and U.S. patent application Ser. No. 12/408,116, entitled “Phthalocyanine Salt Formulations,” the disclosures of which are incorporated by reference herein. Metal phthalocyanines and phthalocyanines bearing axial ligands in particular have been used as photosensitizing agents as a result of their capacity for redox chemistry. Metal phthalocyanines include a diamagnetic metal ion moiety that is either coordinated or covalently bound to the phthalocyanine core. The metal ion can be selected from aluminum (Al), germanium (Ge), gallium (Ga), tin (Sn), zinc (Zn) and silicon (Si) or any other suitable diamagnetic metal ion. Phthalocyanines bearing an axial ligand include phthalocyanine compounds with modifying moieties linked to the central metal.

Representative phthalocyanine compounds include compounds generally characterized by the following formula (I), or a pharmaceutically acceptable salt thereof

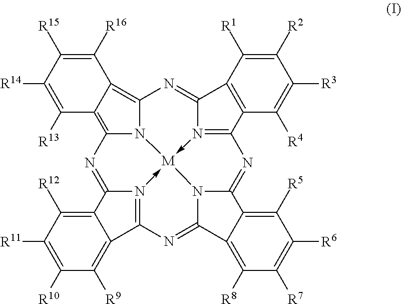

Phthalocyanine compounds include 16 points along the circumference of the fused ring structure that can be represented by R¹-R¹⁶. These positions can be substituted with a wide variety of functional groups. For example, R¹-R¹⁶ can each be independently selected from hydrogen, halogen, nitro, cyano, hydroxy, thiol, amino, carboxy, aryl, heteroaryl, carbocyclyl, heterocyclyl, C₁₋₂₀alkyl, C₁₋₂₀alkenyl, C₁₋₂₀alkynyl, C₁₋₂₀alkoxy, C₁₋₂₀acyl, C₁₋₂₀alkylcarbonyloxy, C₁₋₂₀aralkyl, C₁₋₂₀hetaralkyl, C₁₋₂₀carbocyclylalkyl, C₁₋₂₀heteroyclylalkyl, C₁₋₂₀aminoalkyl, C₁₋₂₀alkylamino C₁₋₂₀thioalkyl, C₁₋₂₀alkylthio, C₁₋₂₀hydroxyalkyl, C₁₋₂₀alkyloxycarbonyl, C₁₋₂₀alkylaminocarbonyl, C₁₋₂₀alkylcarbonylamino, and C_{1-10alkyl}-Z—C₁₋₁₀alkyl. Z is selected from S, NR¹⁷, and O, and if present R¹⁷ is selected from hydrogen, C₁₋₂₀acyl, C₁₋₂₀alkyl, and C₁₋₂₀aralkyl.

Additional embodiments of the invention can use phthalocyanine compounds in which the substituents along the circumference are divided into two groups. Substituents on fused ring structures can be peripheral or non-peripheral substituents. A non-peripheral substituent, as defined herein, is a substituent which is adjacent (i.e., α) to the point of fusion between an outer phenyl ring and an inner pyrrole ring, as found in phthalocyanine compounds as exemplified by Formula I herein. A substituent is peripheral, on the other hand, when it is not a non-peripheral substitutent. For example, in Formula I provided herein, the substituents R², R³, R⁶, R⁷, R¹⁰, R¹¹, R¹⁴, and R¹⁵ are peripheral substituents.

In addition embodiments, the peripheral substituents R¹, R⁴, R⁵, R⁸, R⁹, R¹², R¹³, and R¹⁶ are each independently selected from hydrogen, halogen, nitro, cyano, hydroxy, thiol, amino,and methyl; and R², R³, R⁶, R⁷, R¹⁰, R¹¹, R¹⁴, and R¹⁵ are each independently selected from hydrogen, halogen, nitro, cyano, hydroxy, thiol, amino, carboxy, aryl, heteroaryl, carbocyclyl, heterocyclyl, C₁₋₆alkyl, C₁₋₆alkenyl, C₁₋₆alkynyl, C₁₋₆alkoxy, C₁₋₆acyl, C₁₋₆alkylcarbonyloxy, C₁₋₆carbocyclylalkyl, C₁₋₆aminoalkyl, C₁₋₆alkylamino, C₁₋₆thioalkyl, C₁₋₆alkylthio, C₁₋₆hydroxyalkyl, C₁₋₆alkyloxycarbonyl, C₁₋₆alkylaminocarbonyl, and C₁₋₆alkylcarbonylamino.

Within the center of the fused ring structure of the phthalocyanine compound of formula I is M. M is a diamagnetic metal ion optionally complexed with or covalently bound to one or two axial ligands, wherein the metal ion is coordinated to the phthalocyanine moiety.

In a number of embodiments, the axial ligand M is (G)ₐY[(OSi(CH₃)₂(CH₂)_{b}N_{c}(R′)_{d}(R″)ₑ)_{f}X_{g}]ₚ; wherein Y is selected from Si, Al, Ga, Ge, Zn, or Sn; R′ is selected from H, CH₃, C₂H₅, C₄H₉, C₄H₈NH, C₄H₈N, C₄H₈NCH₃, C₄H₈S, C₄H₈O, C₄H₈Se, OC(O)CH₃, OC(O), CS, CO, CSe, OH, C₄H₈N(CH₂)₃CH₃, (CH₂)₂N(CH₃)₂, (CH₂)ₙN((CH₂)ₒ(CH₃))₂, and an alkyl group having from 1 to 12 carbon atoms; R″ is selected from H, SO₂CH₃, (CH₂)₂N(CH₃)₂, (CH₂)₁₁CH₃, C(S)NHC₆H₁₁O₅, (CH₂)ₙN((CH₂)ₒ(CH₃))₂, and an alkyl group having from 1 to 12 carbon atoms; G is selected from OH and CH₃; a is 0 or 1; b is an integer from 2 to 12; c is 0 or 1; d is an integer from 0 to 3; e is an integer from 0 to 2; f is 1 or 2; g is 0 or 1; n is an integer from 1 to 12; o is an integer from 1 to 11; and p is 1 or 2.

Certain embodiments of the invention provide phthalocyanine compounds having an axial ligand carrying or terminating in an amine function or a quaternary ammonium function attached to the central metal. This is represented in the formula by —N_{c}(R′)_{d}(R″)ₑ in the axial ligand. This amine is believe to provide axial ligands that give the phthalocyanine compounds the capability to bind to various species that assist in transporting the compound to a from their targets within the cell, as well as enhancing the potential for these phthalocyanine compounds to bind to target cells. These compounds are generally much more effective in producing photodynamic activity, apparently as a result of their increased cellular uptake.

Further embodiments provide additional limitations on the structure of the phthalocyanine compound. In some embodiments, Y is Si, while in other embodiments R¹-R¹⁶ are H. In additional embodiments, G is OH and N_{c}(R′)_{d}(R″)ₑ of the axial ligand M is N(CH₃)₂. A preferred phthalocyanine is “Pc 4”, which is a compound having a structure of Formula (I), wherein M is HOSiPcOSi(CH₃)₂(CH₂)₃N(CH₃)₂ and R¹-R¹⁶ are H.

The axial ligand M includes Xg which represents a counterion to the amine group in salt forms of the axial ligand. Accordingly, if X is present (i.e., g is not 0), X is selected from a pharmaceutically acceptable anion and the adjacent amine of the axial ligand will be a quaternary amine having a positive charge. Together the anion and quaternary amine form the salt. The salts chosen for this work can be selected from those formed by acids giving physiologically ubiquitous ions or intermediate metabolite ions in biochemical pathways, such as the acids designated as Class 1 by Stahl (Handbook of Pharmaceutical Salts; Stahl, P. H.; Wermuth, C. G. Eds; Wiley-CH: New York, 2002; p 9-18), or those formed by acids showing little toxicity and good tolerability.

A method of photodynamic treatment of a fungal infection is described herein. Treat”, “treating”, and “treatment”, etc., refer to any action providing a benefit to a subject afflicted with a fungal infection, including improvement in the condition through lessening or suppression of at least one symptom, or delay in progression of the disease, etc. In one embodiment of treatment, administration of the compounds is effective to eliminate the fungal infection; in another embodiment, administration of the phthalocyanine compound is effective to decrease the severity of the fungal infection. Prevention or prophylaxis, on the other hand, refers to either a complete avoidance or delay in the onset of the fungal infection.

The present invention provides a method of photodynamic treatment of a fungal infection. Fungal infection is caused when a fungal spores from a disease-causing fungi enter the body or come into contact with the skin and begin to multiply. Disease-causing fungi may enter the body through the skin, nose, vagina, nails, or mouth. Fungi cause different diseases depending on the specific type of fungus and where they are in the body. Examples of common fungal infections include athlete's foot, candidiasis, histoplasmosis, jock itch (tinea cruris), onychomycosis, fungal paronychia, and tinea versicolor. A genus of fungi that commonly causes fungal infections is Candida. Fungal infections of the skin and nails can also be referred to as dermatophyte infections. Dermatophytes are fungi that require keratin for growth. See Hainer, B., Am Fam Physician., 67(1): 101-8 (2003). Examples of dermatophyte infections (i.e., dermoatophytoses) include tinea capitis, tinea corporis, tinea barbae, tinea faciei, tinea manuum, tinea cruris, tinea pedis, tinia unguium. Common genus causing dermatophytoses are Microsporum, Trichophyton, and Epidermophyton. Dermotophytoses can be diagnosed using various techniques, such as potassium hydroxide microscopy, Wood's lamp examination, fungal culture, and skin or nail biopsy, as understood by those skilled in the art.

The methods described herein are also useful for the treatment of onychomycosis. Tinia unguium is a subset of the disease onychomycosis, which can also be caused by yeast and non-dermatophyte fungi. Onychomycosis is an infection of the nails, typically toe nails, in which the nail plate can have a thickened, yellow, or cloudy appearance, and the nails can become rough and crumbly, or can separate from the nail bed. There are four main types of onychomycosis. These include Distal subungual onychomycosis, which involves invasion of the nail bed and the underside of the nail plate and is usually caused by Trichophyton rubrum; white superficial onychomycosis (WSO), which involves invasion of the superficial layers of the nail plate to form “white islands” on the plate; proximal subungual onychomycosis, which is fungal penetration of the newly formed nail plate through the proximal nail that is most commonly found when the patient is immunocompromised; and candidal onychomycosis, which is a Candida species (e.g., Candida albicans) invasion of the fingernails that is usually prompted by prior damage of the nail by infection or trauma. Onychomycosis can be diagnosed using potassium hydroxide microscopy, fungal culture, or periodic acid-Schiff staining

The term “subject” for purposes of treatment includes any human or animal subject who has a fungal disease or condition amenable to treatment with the phthalocyanine compounds of the present invention. For methods of prevention the subject is any human or animal subject, and in some embodiments the subject is a human who is at risk of developing a fungal infection. The subject may be at risk due to stress, immunosuppression, exposure to the fungal pathogen, advanced age, and so on. Besides being useful for human treatment, the compounds of the present invention are also useful for veterinary treatment of mammals, including companion animals and farm animals, such as, but not limited to dogs, cats, horses, cows, sheep, and pigs.

In some embodiments of the invention, the phthalocyanine compound includes a free base (e.g., an amine site) and which associates with an acid to form a salt. Thus, suitable pharmaceutically acceptable salts of phthalocyanine compounds may be prepared from an inorganic acid or from an organic acid. Examples of such inorganic acids are hydrochloric, hydrobromic, hydrofluoric, hydroiodic, nitric, carbonic, sulfuric, and phosphoric acid. Appropriate organic acids may be selected from aliphatic, cycloaliphatic, aromatic, araliphatic, heterocyclic, carboxylic, and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicylic, p-hydroxybenzoic, phenylacetic, mandelic, ambonic, pamoic, methanesulfonic, ethanesulfonic, benzenesulfonic, pantothenic, 2-hydroxyethanesulfonic, toluenesulfonic, sulfanilic, cyclohexylaminosulfonic, stearic, algenic, siloxane, β-hydroxybutyric, galactaric, and galacturonic acids. Generally, the acid forms a counterion upon associating with amines of the phthalocyanine Examples of preferred counter ions include chloride, bromide, nitrate, sulfate, tosylate, phosphate, tartrate, and maleate. Another set of suitable counter ions includes malate, mesylate, inosate, dimethylphosphonate, methylsulfonate, and sulfonate anions.

The phthalocyanine compound can be administered in a pharmaceutical carrier. A “Pharmaceutically acceptable carrier,” as used herein, means a pharmaceutically acceptable material, composition or vehicle, such as a liquid or solid filler, diluent, excipient, solvent or encapsulating material, that improves the ability of the phthalocyanine compound to be used for treatment. For example, the pharmaceutical carrier can improve the storage characteristics of the phthalocyanine compound, or it can improve its ability to be delivered to a target tissue. Each carrier must be “acceptable” in the sense of being compatible with the other ingredients of the formulation and not injurious to the patient. An example of a pharmaceutically carrier suitable for delivery of a phthalocyanine compound to skin or nail tissue is a topical formulation.

In some embodiments, the phthalocyanine compound is applied as part of a topical formulation. Topical administration of the phthalocyanine compound can be accomplished using various different formulations such as powders, sprays, ointments, pastes, creams, lotions, gels, solutions, or patches. The phthalocyanine compound may be mixed under sterile conditions with a pharmaceutically acceptable carrier, and with any preservatives, buffers, or propellants which may be required. The ointments, pastes, creams, solutions, foams, lacquers, oils and gels may contain excipients in addition to the phthalocyanine compound active ingredient. An example of a suitable formulation for topical delivery of a phthalocyanine compound is a 70% ethanol and 30% propylene glycol solution.

Examples of topical formulations include ointments and creams. Ointments are homogeneous, semi-solid preparations intended for external application to the skin or mucous membranes. They are used as emollients or for the application of active ingredients to the skin for protective, therapeutic, or prophylactic purposes and where a degree of occlusion is desired. Ointments can be formulated using hydrophobic, hydrophilic, or water-emulsifying bases to provide preparations for various applications. Creams, on the other hand, are semi-solid emulsions; i.e., a mixture of oil and water. They are divided into two types: oil-in-water creams which are composed of small droplets of oil dispersed in a continuous aqueous phase, and water-in-oil creams which are composed of small droplets of water dispersed in a continuous oily phase.

The method of photodynamic treatment includes administering a therapeutically effective amount of a phthalocyanine compound. A “therapeutically effective amount” of a phthalocyanine compound for the method of treatment refers to an amount of the compound(s) in a preparation which, when administered as part of a desired dosage regimen, alleviates a symptom, ameliorates a condition, or slows the onset of disease conditions according to clinically acceptable standards for the disease or condition to be treated or the cosmetic purpose, e.g., at a reasonable benefit/risk ratio applicable to any medical treatment.

Dosage amounts and preferred formulations can be readily established by reference to known treatment or prevention regimens. The amount of phthalocyanine compound that is administered and the dosage regimen for treating a fungal infection with a phthalocyanine compound using the method of this invention depends on a variety of factors, including the age, weight, sex, and medical condition of the subject, the severity of the disease, the site and frequency of administration, the character of the skin to which the agent is applied, and the particular compound employed, and thus may vary widely. Such treatments may be administered as often as necessary and for the period of time judged necessary by the treating physician. One of skill in the art will appreciate that the dosage regime or therapeutically effective amount of the inhibitor to be administrated may need to be optimized for each individual. The topical formulations may contain active ingredient in the range of about 0.001 to 1 mg/ml, preferably in the range of about 0.01 and 0.1 mg/ml and most preferably between about 0.05 mg/ml to about 0.1 mg/ml. Suitable amounts vary depending on the phthalocyanine compound being used, but can be readily determined by one skilled in the art.

The area of tissue to which the phthalocyanine compound is applied can vary depending on the nature of the disease or condition be treated or prevented. Typically, treatment is confined to the area in which the disease or abnormal condition is exhibited, or where the disease or condition is expected to occur in the case of prophylactic treatment. Examples of tissues that can be treated include the toenails, fingernails, and skin. Areas of skin that can be treated include portions of skin on the head, arms, legs, and torso.

The phthalocyanine compound can be administered to the area of tissue immediately before photoirradiation is delivered, or the photoirradiation can be delayed to provide some time for the phthalocyanine compound to be taken up by the tissue. For example, the photoirradiation can be administered from 0.5 to 5 minutes after administration of the phthalocyanine compound to the tissue. The phthalocyanine compound can be administered in pure form, applied after being dissolved in a non-toxic solvent, or it can be administered in a formulation (e.g., a topical formulation). The solution including the phthalocyanine compound can be administered to the area of tissue by a suitable device such as a syringe or pipette, and can then be manually spread over the area of tissue (e.g., by a gloved finger). Alternately, the solution including the phthalocyanine compound can be applied to an area of tissue via a spray.

The method of treatment described herein includes administering photoirradiation with light, i.e., photodynamic treatment, having a wavelength suitable to activate the phthalocyanine compound to the area of tissue. Photoirradiation, as used herein, refers to shining light for a discrete period of time, typically only a few seconds or minutes. For example, one can administer photoirradiation for about 1 to 30 minutes or from 5 to 20 minutes. The duration of photoirradiation generally varies inversely with the intensity of the photoirradiation delivered, in order for the photoirradiation to provide a specific amount of light energy. The light should be administered for a period of time and intensity sufficient to activate the photosensitizing agent. For a phthalocyanine compound, 10 to 20 J/cm² of energy will typically be sufficient to activate the compound. However, amounts ranging from about 5 to about 200 J/cm² can be used in some embodiments of the invention.

The light can be provided by a laser, light emitting diode, or other light source known to those skilled in the art. The light can be provided by a single source, or a plurality of light sources can be used. In the case of internal tissue, it is necessary to either provide access to the tissue for the light source, or to provide an arthroscopic light source. For example, the tissue can first be exposed through surgery, or the light can be delivered through an optical cable. When treating internal tissue, it may be preferable to administer the phthalocyanine compound systemically, or it may be preferable to apply a topical formulation to the internal tissue once it has been exposed.

### Definitions

Unless otherwise defined, all technical and scientific terms used herein have the same meaning as commonly understood by one of ordinary skill in the art to which this application pertains. Although methods and materials similar or equivalent to those described herein can be used in the practice or testing of the exemplary embodiments, suitable methods and materials are described below. In case of conflict, the present specification, including definitions, will control. In addition, the materials, methods, and examples are illustrative only and not intended to be limiting.

Unless otherwise specified, “a,” “an,” “the,” and “at least one” are used interchangeably and mean one or more than one. Also herein, the recitations of numerical ranges by endpoints include all numbers subsumed within that range (e.g., 1 to 5 includes 1, 1.5, 2, 2.75, 3, 3.80, 4, 5, etc.).

It should be understood that all units of measurement provided herein can be readily substituted for alternate units known by those skilled in the art, and that the corresponding numbers should be adjusted to take into account the conversion from one unit to another. For examples, measurement of fluence in milliwatt/cm² can be replaced with Joules/cm².

The term “C_{x-y}acyl” refers to a group represented by the general formula: C_{x-y}alkyl-C(O)—

The term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-tirfluoroethyl, etc. C₀ alkyl indicates a hydrogen where the group is in a terminal position, a bond if internal. The terms “C₂-yalkenyl” and “C₂-yalkynyl” refer to substituted or unsubstituted unsaturated aliphatic groups analogous in length and possible substitution to the alkyls described above, but that contain at least one double or triple bond respectively. When such alkenyl or alkynyl groups include more than one unsaturated bond, they can be referred to as polyunsaturated alkenyl or alkynyl groups.

The term “alkoxy” refers to an alkyl group having an oxygen attached thereto. Representative alkoxy groups include methoxy, ethoxy, propoxy, tert-butoxy and the like. An “ether” is two hydrocarbon groups covalently linked by an oxygen. Accordingly, the substituent of an alkyl that renders that alkyl an ether is or resembles an alkoxy.

The terms “amine” and “amino” are art-recognized and refer to both unsubstituted and substituted amines and salts thereof.

The term “aryl” as used herein includes 5-, 6-, and 7-membered substituted or unsubstituted single-ring aromatic groups in which each atom of the ring is carbon. Aryl groups include benzene, phenol, aniline, and the like.

The terms “carbocycle” and “carbocyclyl”, as used herein, refer to a non-aromatic substituted or unsubstituted ring in which each atom of the ring is carbon.

The terms “heteroaryl” includes substituted or unsubstituted aromatic 5- to 7-membered ring structures, more preferably 5- to 6-membered rings, whose ring structures include one to four heteroatoms. Heteroaryl groups include, for example, pyrrole, furan, thiophene, imidazole, oxazole, thiazole, triazole, pyrazole, pyridine, pyrazine, pyridazine and pyrimidine, and the like.

The term “heteroatom” as used herein means an atom of any element other than carbon or hydrogen. Preferred heteroatoms are nitrogen, oxygen, phosphorus, and sulfur.

The terms “heterocyclyl” or “heterocyclic group” refer to substituted or unsubstituted non-aromatic 3- to 10-membered ring structures, more preferably 3- to 7-membered rings, whose ring structures include one to four heteroatoms.

The term “substituted” refers to moieties having substituents replacing a hydrogen on one or more carbons of the framework. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The following examples are included for purposes of illustration and are not intended to limit the scope of the invention.

### EXAMPLES

### Example 1

### Photodynamic Therapy with Pc 4 for Fungal Infections

The inventors carried out experiments to obtain data on Pc 4 PDT induced cytotoxicity on planktonic C. albicans. In addition, confocal imaging data was obtained demonstrating that Pc 4 is effectively taken up by the C. albicans into cytosolic compartments, but not in the plasma membrane/cell wall or nucleus, similar to the Pc 4 distribution observed in mammalian cells.

### Materials and Experimental Design:

Cell growth conditions: A single colony of C. albicans (SC-5134) strain was obtained by inoculation and incubating with 25 ml in Yeast Nitrogen Base (YNB) medium (Becton Dickinson, New Jersey, USA) supplemented with 50 mmol/L dextrose, anhydrous (Fisher Scientific, Pittsburgh, USA), henceforth referred to as standard growth medium, for at least 5 hours in a shaking incubator at 37° C. These cells would then be used to examine the subcellular localization of Pc 4, cell death and survival assays following Pc 4-PDT treatment, via confocal microscopy, XTT and CFU assay, respectively.

Confocal Microscopy: To investigate the uptake and localization of Pc 4, cells were loaded with 200 nM of Pc 4 in the presence of 10% FBS in the standard growth medium for 16 to 18 hours. 30 minutes prior to imaging Pc 4, 50 nM of MitoTrackerRed (Invitrogen, Carlsbad, Calif., USA) and/or 50 μg/mL AlexaFluor 488 conjugated Concanavalin A (ConA) (Invitrogen, Carlsbad, Calif., USA) and/or 10 μg/ml Hoechst 33342 (Invitrogen, Carlsbad, Calif., USAS) were loaded to the cells. 5 to 10 μL of stained cells were then placed on a slide with a glass coverslip and all images were acquired using the UltraVIEW VoX spinning disk confocal system (PerkinElmer, Waltham, Mass., USA) with a Leica DMI6000B microscope (Leica Microsystems, Inc., Bannockburn, Ill., USA), which was equipped with a HCX PL APO 100×/0.4 oil immersion objective. Confocal images of Pc 4 fluorescence, MitoTrackerRed, ConA and Hoechst were collected using solid state diode lasers, with 640-nm, 561-nm, 488-nm and 405-nm excitation light, respectively, and with appropriate emission filters.

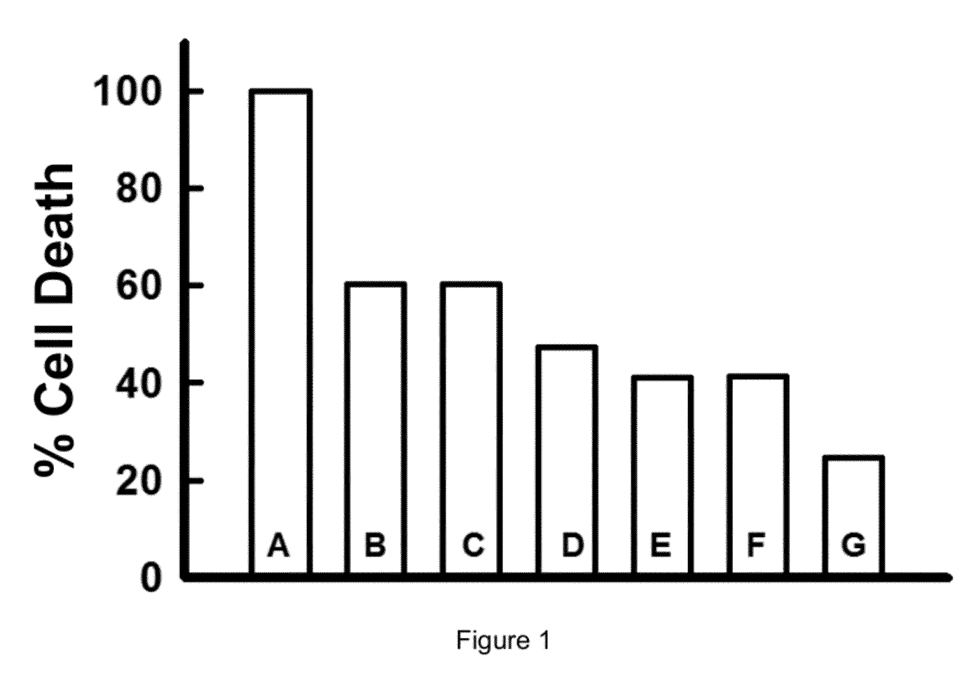

Metabolic Activity Assay: XTT reduction assays were performed according to the procedure of Chandra et al. Chandra et al., Nature Protocol. 3(12): 1909-2 (2008). Briefly, in the dark, aliquots of 2 ml of cells were treated with 200 nM of Pc 4 in 10% FBS standard growth medium in a 37° C. shaker. The next day, the cells were photoirradiated with red light using a light-emitting diode array (EFOS, Mississauga, Ontario, Canada) at a fluence of 200, 500, 1000 and 2000 mil/cm² (1 milliwat/cm², ymax ˜670-675 nm) at room temperature. For negative controls, a 2 ml aliquot was not photoirradiated and a 2 ml aliquot simply was loaded overnight with an appropriate amount of N′,N′-dimethylformamide and then photoirridated with the highest fluence the next day. For positive control, either cells were treated with 30% ethanol or 1 uM of Thapsigargin. XTT (Sigma-Aldrich Corp., St. Louis, Mo.) was dissolved in PBS at a final concentration of 5 mg/ml. The solution was filter-sterilized and stored frozen at −70° C. until use. Menadione solution (Sigma-Aldrich Corp, St. Louis, Mo.) was initially dissolved in acetone at a stock concentration of 10 mM and further diluted at a final concentration with 1× PBS of 1 mM. After light-treatment alone, Pc 4 alone and different doses of Pc 4 PDT treatments, cells were centrifuged at 1,500 RPM and resuspended in 2 ml of 1× PBS. One ml of each treatment condition was used to incubate with XTT (final concentration 1 mg/ml) and menadione (final concentration 1 mM) for 5 hours in the dark and then transferred to 1 ml plastic cuvette for absorbance measurement. Colorimetric changes, which reflect the viability of the cells given different treatment conditions, were measured at 492-nm using a spectrophotometer (Spectronic Genesys™ 5, Analytical Instrument, Golden Valley, Minn., USA). The results are shown in FIG. 1.

Colonies Formation Assay: Briefly, the 1 ml of each treatment condition was diluted into 1:10,000, 1:50,000 or 1:100,000 and plated 100 μL onto Sabouraud dextrose agar plate (Becton Dickinson, New Jersey, USA). All plates were incubated at 37° C. and colonies were imaged and counted the following day for each plate/condition.

### Results:

Pc 4 0.1 mg/ml solution is able to penetrate the cell membrane of C. albicans and can be seen within the cytosol. The confocal images indicated no colocalization with the nuclear stain Hoechst and the cell wall stain ConA. There was partial colocalization with mitotracker indicating Pc 4 binds to mitochondrial membranes and other cytosolic components.

Significance: The data demonstrate that Pc 4-PDT can be utilized for antifungal treatment. Pc 4-PDT may offer additional advantages over conventional antifungal drugs because it does not affect the fungal genome and thus development of resistant strains is minimized. The mode of cell killing is probably similar to Pc 4-PDT killing of mammalian cells which is mainly via apoptosis, which is triggered by damage secondary to reactive oxygen species formation. C. albicans, in particular, has been shown to have apoptotic mechanisms homologous to mammalian cells. Pc 4-PDT can therefore be developed as an alternative novel therapy for candidal and possibly, other fungal infections.

### Example 2

### Photodynamic Therapy with Pc 4 Induces Apoptosis of Candida albicans

The in vitro cytotoxicity and mechanism of PDT, using the photosensitizer Pc 4, was assessed in planktonic C. albicans. Confocal image analysis confirmed that Pc 4 penetrates the C. albicans cell membrane and localizes to cytosolic organelles, including mitochondria. A colony formation assay showed that 1.0 μM Pc 4 followed by light at 2.0 J/cm² reduced cell survival by 4 logs. XTT (2,3-bis [2-Methoxy-4-nitro-5-sulfophenyl]-2H-tetrazolium-5-carboxyanilide inner salt) assay revealed that Pc 4-PDT impaired fungal metabolic activity, which was confirmed using the FUN-1 (2-chloro-4-[2,3-dihydro-3-methyl-(benzo-1,3-thiazol-2-yl)-methylidene]-1-phenylquinolium iodide)fluorescence probe. Furthermore, changes in the nuclear morphology characteristic of apoptosis were observed which were substantiated by the increased externalization of phosphatidylserine and DNA fragmentation following Pc 4-PDT. These data indicate that Pc 4-PDT can induce apoptosis in C. albicans.

### Materials and Methods

Organism and Culture Conditions: C. albicans (strain SC-5134) was grown in yeast nitrogen base (YNB) medium (Difco Laboratories, Detroit, Mich., USA) supplemented with 50 mM glucose and incubated for 24 hours at 37° C. in an orbital water bath shaker at 150 rpm. Cells were then harvested and washed twice with 0.15 M phosphate-buffered saline (PBS; pH 7.2, calcium- and magnesium-free). Cells were re-suspended in 10 mL of 1× PBS, further serial diluted, counted using a hemocytometer, standardized and used immediately. Chandra et al., Nat Protoc 3:1909-1924 (2008).

Confocal Microscopy: To investigate the uptake and localization of Pc 4, cells in standard growth medium plus 10% Fetal Bovine Serum (FBS) were loaded with 0.2 μM Pc 4 and incubated for 16 to 18 hr. Thirty minutes prior to imaging Pc 4, 50 nM MitoTracker Red (Invitrogen, Carlsbad, Calif., USA) or 50 μg/mL AlexaFluor 488-conjugated Concanavalin (Con) A (Invitrogen, Carlsbad, Calif., USA) and/or 10 μg/mL Hoechst 33342 (Invitrogen, Carlsbad, Calif., USA) was loaded onto cells. Five to 10 μL of stained cells were placed on a slide with a glass coverslip and all images acquired using an UltraVIEW VoX spinning disk confocal system (PerkinElmer, Waltham, Mass., USA), which was mounted on a Leica DMI6000B microscope (Leica Microsystems, Inc., Bannockburn, Ill., USA) equipped with a HCX PL APO 100×/1.4 oil immersion objective. Confocal images of Pc 4 fluorescence, MitoTracker Red, ConA (or FITC-labeled Annexin V) and Hoechst 33342 were collected using solid-state diode lasers, with 640-nm, 561-nm, 488-nm and 405-nm excitation light, respectively, and with appropriate emission filters.

Pc 4-Photodynamic Treatment Conditions: The phthalocyanine photosensitizer Pc 4 was kindly provided by Dr. Malcolm E. Kenney (Department of Chemistry, Case Western Reserve University, Cleveland, Ohio, USA). Stock solutions (0.5 mM) of Pc 4 were prepared in N′,N′-dimethylformamide (DMF, ThermoFisher, Waltham, Mass., USA) and stored at 4° C. in the dark. C. albicans cultures were incubated with Pc 4 concentrations ranging from 0.1 to 1.0 μM in 1× PBS containing 10% FBS for at least 1 hr at 37° C. in the dark and subsequently irradiated with red light using a light-emitting diode array (EFOS, Mississauga, Ontario, Canada) at a fluence ranging from 1.0 to 2.0 J/cm² (1.0 mW/cm², λₘₐₓ ˜670-675 nm) at room temperature.

Colony forming unit (CFU) phototoxicity assay: CFU assay was done as described by Chandra et al. with minor modifications. Chandra et al., Nat Protoc 3:1909-1924 (2008). Step A was first carried out. Briefly, following photoirradiation, C. albicans cell suspensions that received various Pc 4-PDT doses plus dark controls were serially diluted in 1× PBS then plated on Sabouraud dextrose agar (Becton, Dickinson and Company, Sparks, Md., USA) plates and incubated at 37° C. to allow colony formation. Aliquots of cell suspension were plated onto the 100-mm Petri dishes in amounts sufficient to yield 50-100 colonies per dish. After 24 hours, colonies were counted by eye, and all the percentage of surviving cells from the Pc 4-PDT cultures was normalized against that of cultures exposed to Pc 4 or light alone. In order to qualitatively assess C. albicans survivability post-treatment and show the results in one concise image, all yeast samples with varying concentrations were plated on one 100-mm petri dish for step B. All cell suspensions were prepared as in (A). One 100-mm petri dish with Sabouraud dextrose agar was divided into eight equal wedges. After Pc 4-PDT, one drop of each cell suspension as well as light and dark controls were placed on each wedge and incubated for 24 hours at 37° C.

Metabolic assays: (A) XTT—One hour following photo-irradiation of Pc 4-loaded cells, the metabolic activity of the C. albicans was assayed using the colorless sodium salt of XTT (Sigma-Aldrich, St. Louis, Mo., USA), which is converted by mitochondrial dehydrogenases of viable cells into a water-soluble orange formazan derivative through the reduction of the tetrazolium ring of XTT. The absorbance of the resulting orange solution was measured using a spectrophotometer (Spectronic Genesys 5, Analytical Instrument, Golden Valley, Minn., USA) at 492 nm. (B) FUN-1 (Invitrogen, Carlsbad, Calif., USA) is a fluorescent probe used to assess yeast metabolic activity. Metabolically active yeast will endocytose FUN-1 and process it into an orange-red cylindrical intravacuole structure. One hour following Pc 4-PDT treatment, cells were loaded with 10 μM FUN-1 for 30 minutes at 37° C. Cells were washed before placing 10 μL between a slide with a cover slip and confocal images were acquired with an UltraVIEW VoX spinning disc confocal system (PerkinElmer), which is mounted on a Leica DMI6000B microscope (Leica Microsystems) equipped with a HCX PL APO 100×/1.4 oil immersion objective. FUN-1 fluorescence was excited using solid state diode lasers (488 nm) and collected with a 580-650 nm band pass emission filter.

Apoptosis Analyses: (A) FITC-labeled Annexin V staining—To assess the externalization of phosphatidylserine, protoplasts of C. albicans were stained with FITC-labeled Annexin V using the Annexin V-FLUOS staining kit (Roche, Germany) with minor modifications. Madeo et al., J Cell Biol 139:729-734 (1997); Park et al., Biochem Biophys Res Commun 394:170-172 (2010). Briefly, control and treated cells were washed three times with 1× PBS before incubating at 37° C. for 30 min in a protoplasting solution (50 mM KH₂PO₄, 5 mM EDTA and 50 mM DTT). Then cells were digested at 37° C. for 45 min in an enzyme solution (50 mM K₂HPO₄, 40 mM 2-mercaptoethanol, 3 μg/mL Chitinase, 1.8 μg/mL Lyticase, 0.15 mg/mL Zymolyase and 2.4 M sorbitol). 1× PBS was added to each sample and incubated for 30 min at room temperature in all experiments. Protoplasts were then incubated with 100 μL of Annexin V-FLUOS labeling solution for 15 min at 25° C. FITC-labeled Annexin V fluorescence was detected by both widefield (Leica DMI6000B microscope; Leica Microsystems, Inc., Bannockburn, Ill., USA) and confocal fluorescence microscopy.

(B) DNA Fragmentation—Following photoirradiation, planktonic C. albicans in growth medium at 10⁶ cells/mL that had been treated with different Pc 4-PDT doses (0.1 to 1.0 μM) or served as dark control (1.0 μM Pc 4 without light) or light control (vehicle with light), and those treated with 20 mM hydrogen peroxide (ThermoFisher Scientific, Waltham, Mass., USA) were pelleted, resuspended in 1× PBS and placed on a 37° C. orbital shaker overnight. Subsequently, C. albicans total chromosomal DNA was extracted using the MasterPure™ Yeast DNA Purification Kit (Epicentre Biotechnologies, Madison, Wis., USA) per the manufacturer's protocol. Extracted DNA concentrations were determined with a spectrophotometer (Nanodrop™ 3000, ThermoFisher Scientific, Waltham, Mass., USA) at a wavelength range of 260-280 nm. DNA (3.0 μg) was loaded onto a 1.0% (w/v) agarose gel (Bioexpress, Kaysbille, Utah, USA) impregnated with ethidium bromide (ThermoFisher, Waltham, Mass., USA) and subjected to electrophoresis. The gel was subsequently visualized using a UV transilluminator (VersaDoc™, Bio-Rad, Hercules, Calif., USA).

### Results

Pc 4 is Localized to Mitochondria—The inventors have previously shown that Pc 4 preferentially, but not exclusively, binds to the mitochondria of a variety of mammalian cells. To determine whether the binding pattern of Pc 4 is similar in fungi, C. albicans was loaded with Pc 4 in glucose-supplemented YNB and examined its fluorescence by confocal microscopy. The majority of Pc 4 fluorescence was detected in the cytoplasmic compartment but not in the cell wall, plasma membrane or nucleus, similar to its distribution in mammalian cells. Trivedi et al., Photochem Photobiol 71:634-639 (2000). In C. albicans, Pc 4 displayed a punctate pattern of fluorescence primarily localized in a cytoplasmic region distinct from the plasma membrane and resembling mitochondria. To test whether mitochondria are sites of localization of Pc 4, cells were co-loaded with MitoTracker Red, a mitochondrion-specific dye. The bright, punctate fluorescence seen in the Pc 4 image usually corresponds to the MitoTracker fluorescence, as indicated in a merged image. The absence of a yellow merged image in some cases may reflect an imbalance in the uptake of the two fluorescent dyes. In addition, as previously found in mammalian cells, Pc 4 also binds to other intracellular organelles, presumably ER membranes, as well as other vesicular membranes.

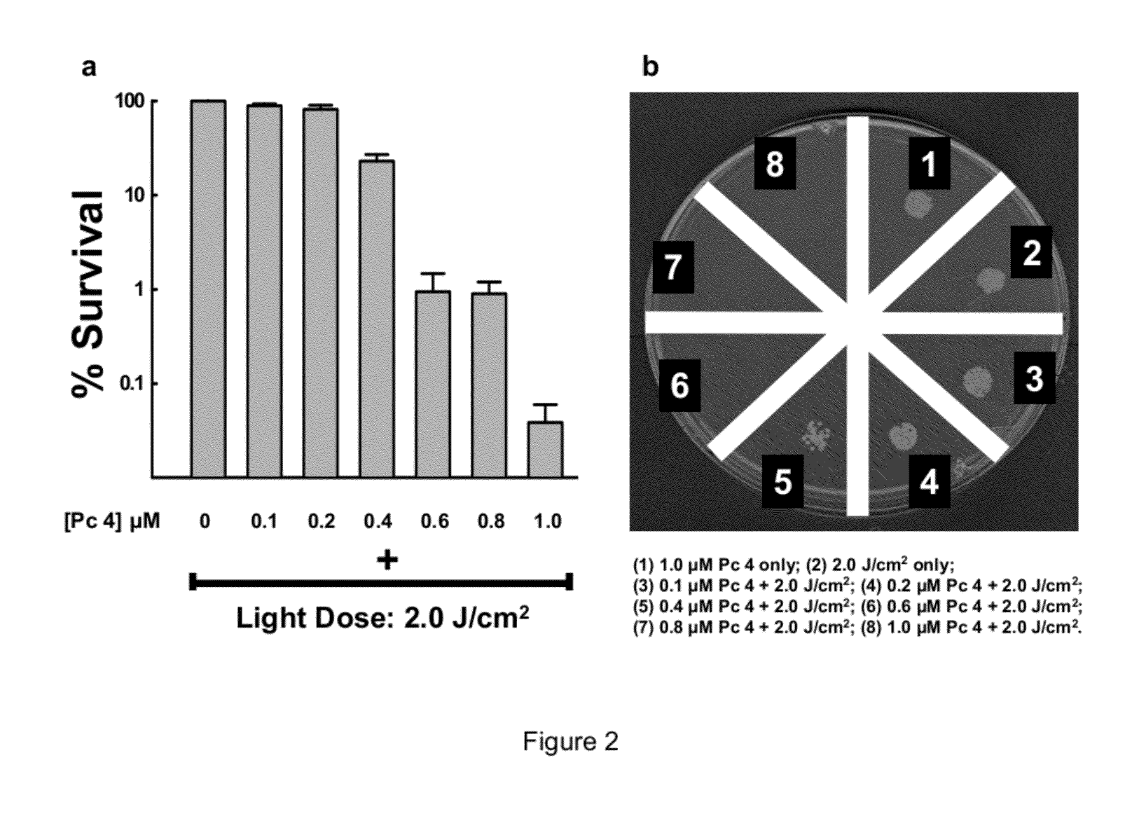

Photodynamic Therapy induces cell death in C. albicans—Having established that Pc 4 localizes to mitochondria as well as other intracellular membranes, the inventors next determined a dose of PDT that would yield several logarithmics of cell killing. C. albicans were loaded with 0.1-1.0 μM Pc 4 for at least 2 hr, then photoirradiated and plated. With 1.0 μM Pc 4 and 2.0 J/cm², the inventors consistently detected 3 to 4 logs of cell killing, as determined by colony forming units (CFU) assay. FIG. 2. In addition, neither the highest dose of photosensitizer alone nor light alone affected cell proliferation and cell death, when compared to untreated cells (data not shown).

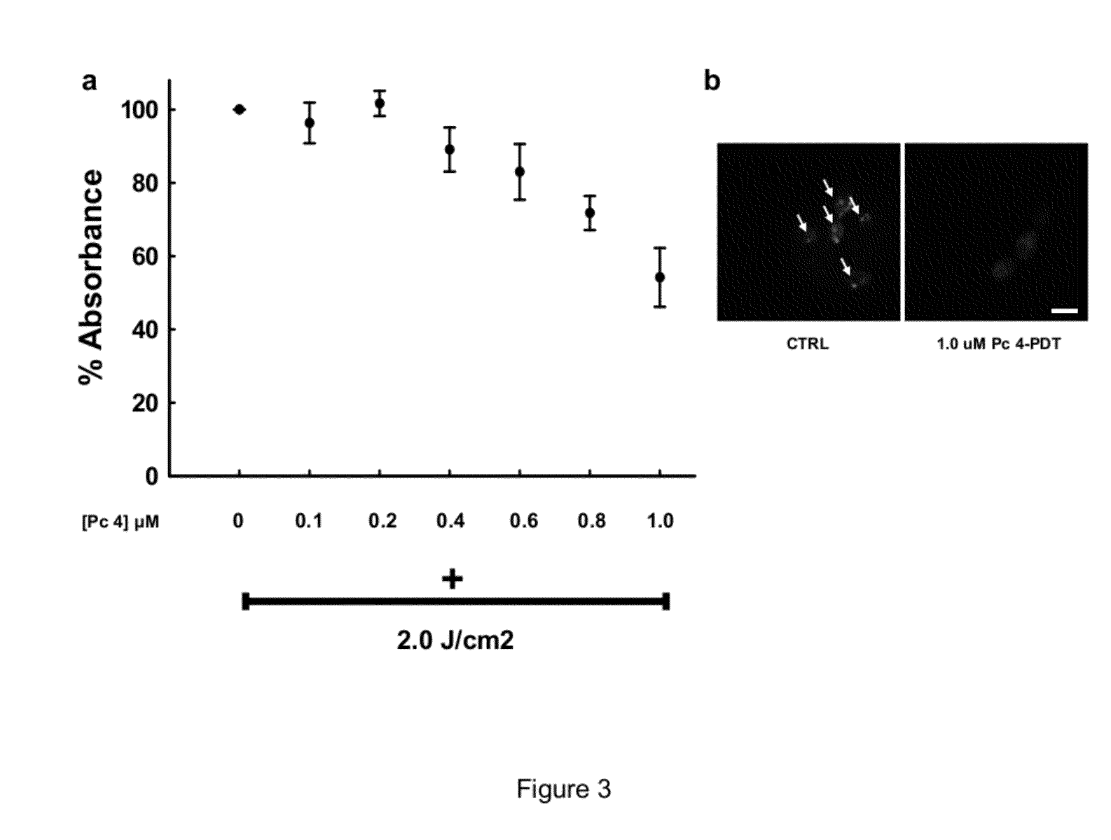

Photodynamic Therapy inhibits metabolic activity in C. albicans—Over the same dose range, the ability of Pc 4-PDT to affect various short-term measures of metabolic activity of the organisms was assessed. Mitochondrial dehydrogenases of viable cells reduce the tetrazolium ring of XTT, a colorless compound, to yield a water-soluble orange formazan derivative. A decrease in absorbance is indicative of a loss of metabolic activity. With the highest Pc 4-PDT dose, metabolic activity was reduced by almost 50% within an hour following photoirradiation of Pc 4-loaded cells. FIG. 3. As an independent indicator of yeast metabolic activity and viability, the fluorescent probe FUN-1 was used. This probe typically enters vacuoles of viable yeast cells and forms cylindrical intravacuole structures that fluoresce orange-green. Millard et al., Appl Environ Microbiol 63:2897-2905 (1997). Since this process requires ATP, non-viable or metabolically inactive cells cannot incorporate FUN-1 and will exhibit minimal or no fluorescence. As shown in the inset confocal microscopy images of FIG. 3, there was much reduced FUN-1 fluorescence in cells exposed to PDT with 1.0 μM Pc 4 as compared to control cells exposed to Pc 4 but not irradiated. This provides further evidence of the loss of metabolic activity and, in turn, yeast viability, after Pc 4-PDT.

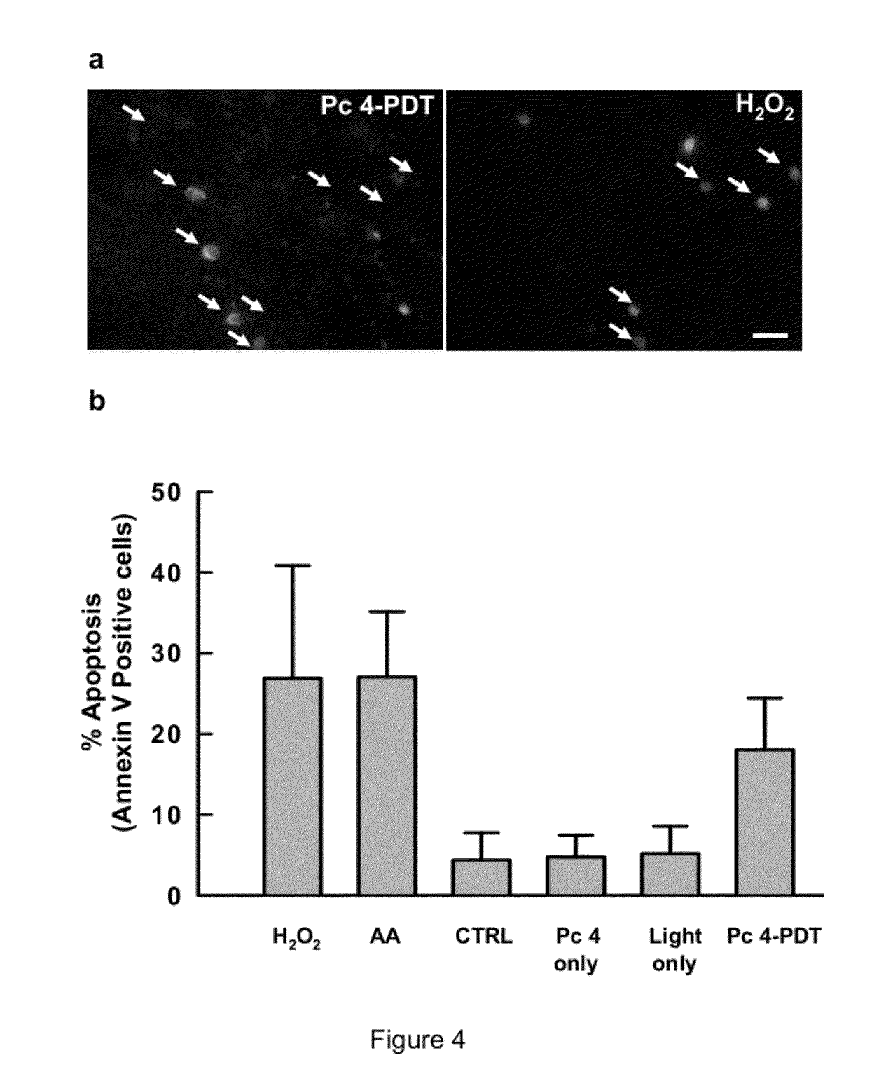

Pc 4-PDT induces apoptosis in C. albicans—Like mammalian cells, yeasts also have an asymmetric distribution of phosphatidylserine (PS) in the lipid bilayer of the plasma membrane. Phillips et al., Proc Natl Acad Sci USA 100:14327-14332 (2003). Since the exposure of PS on the outer surface of the plasma membrane is a common early marker of apoptosis in mammalian cells, the inventors investigated the extent of this phenomenon in Pc 4-PDT-treated C. albicans, as assessed by the binding of FITC-labeled Annexin V. Plasma membrane-associated FITC-Annexin V was detected in protoplasts of cells that had been treated with 1.0 μM Pc 4-PDT, hydrogen peroxide or acetic acid. FIG. 4a. Externalization of PS was readily detected within 4 to 5 hours of PDT (FIG. 4b), when 20% of Pc 4-PDT-treated cells were Annexin V positive, as compared to ˜27% of cells treated with H₂O₂ or acetic acid. Fewer than 5% of negative controls were Annexin V positive.

Chromatin condensation, an indicator of a late stage of apoptosis, was detected by confocal microscopy at the highest dose of Pc 4-PDT 24 hr after Pc 4-PDT or treatment with peroxide. DNA fragmentation is also a late event in apoptosis. DNA gel electrophoresis revealed increasing DNA fragmentation with increasing dose of Pc 4-PDT. At 0.4 μM Pc 4-PDT, there is clear evidence of DNA degradation with faster migration of the DNA. By 0.6 μM Pc 4-PDT, the DNA fragmentation pattern matched that of the hydrogen peroxide positive control with a complete loss of banding and extensive migration of the small DNA fragments.

### Discussion

C. albicans has previously been demonstrated to undergo programmed cell death, or apoptosis, following a variety of treatments, including commonly used anti-fungal agents. Almeida et al., Biochim Biophys Acta 1783:1436-1448 (2008). Although PDT sensitized by Photofrin®, meso-tetra (N-methyl-4-pyridyl) porphine tetra tosylate (TMP-1363) or the porphyrin precursor 5-aminolevulinic acid has been shown to have cytotoxic effects against fungi, the precise mechanism of cell death has not been clearly defined. Chabrier-Rosello et al., J Photochem Photobiol B 99:117-125 (2010). The data provided herein suggest that PDT, sensitized by the phthalocyanine Pc 4, induces apoptosis in C. albicans, exhibiting many similarities with mammalian cells undergoing mitochondrial-mediated programmed cell death.

For PDT to be effective against fungi, the photosensitizer must be able to permeate through their cell wall. Photosensitizing drugs, including Photofrin®, TMP-1363, cationic porphyrin 5-phenyl-10,15,20-Tris(N-methyl-4-pyridyl)porphyrin chloride (TriP[4]) and chlorine (e6) have been demonstrated to target different cellular domains, such as plasma membrane or mitochondria. Irradiation of these photosensiziters has been shown to generate oxidative damage that leads to cytotoxicity. Lambrechts et al., Antimicrob Agents Chemother 49:2026-2034 (2005). In this study, mitochondria is one of the targets that Pc 4 localizes to in C. albicans as evidenced by co-localization experiments as well as by XTT and FUN-1 assays, which indicated that the mitochondrial pathway was involved in Pc 4-PDT induced cell death (FIG. 3).

Sensitivity to photodynamic killing has been found to vary considerably amongst different mammalian cell lines and it may not necessarily be dependent upon a specific subcellular binding of the photosensitizing agent. Xue et al., Exp Cell Res 263:145-155 (2001). Therefore, it was not surprising to find that the effective dose of Pc 4-PDT found in C. albicans differed from doses used to kill tumor cell lines. Compared to mammalian cells, the effective Pc 4-PDT dose for C. ablicans was relatively higher (FIG. 2). In order to produce a significant cytotoxic PDT effect, it was necessary to apply as much as 2.0 J/cm² dose of photoirradiation. The need for higher PDT doses in fungi may be due to the presence of different morphology, i.e., hyphal structure, which could be more recalcitrant to treatment than yeast forms. It has been recognized that modes of cell death in fungi include necrotic, autophagic as well as apoptotic pathways. Eisenberg et al., Apoptosis, 15(3):257-68 (2010). The results from the metabolic activity and clonogenic assays demonstrate that Pc 4-PDT was effective in killing planktonic C. albicans in vitro. In addition, similar to its effects seen in mammalian cells, Pc 4-PDT causes C. albicans to undergo apoptosis as a mode of cell death. The data demonstrate that Pc 4-PDT may have the potential to become an antifungal treatment.

### Example 3

### Pc 4-PDT Treatment of Infections Caused by T. rubrum

Data indicate that Pc 4-PDT can also be used to treat fungal infections caused by T. rubrum. To investigate the uptake and localization of Pc 4, T. rubrum was grown onto Sabouraud potato agar plate for at least 24 hours at 28° C. T. rubrum microconidia, separated from the hyphae cells, were loaded with 200 nM of Pc 4 in the presence of 10% FBS in the standard growth medium at least two hours. Thirty minutes prior to confocal imaging of Pc 4, 10 μg/mL Hoechst 33342 (Invitrogen, Carlsbad, Calif., USA) were loaded to the cells. Five to 10 μL of stained cells were placed onto a slide with a glass coverslip and all images acquired using the UltraVIEWVoX™ spinning disk confocal system (PerkinElmer, Waltham, Mass., USA) which is mounted on a Leica™ DMI6000B microscope (Leica Microsystems, Inc., Bannockburn, Ill., USA) equipped with a HCX PL APO 100×/1.4 oil immersion objective. Confocal images of Pc 4 fluorescence and Hoechst were collected using solid state diode lasers, with 640-nm and 405-nm excitation light, respectively, and with appropriate emission filters. The images obtained confirmed that Pc 4 localizes within the cytosol of T. rubrum. The absence of overlap with Pc 4 and Hoechst suggests that Pc 4 does not bind to the cell nucleus. The pattern of cytosolic binding seen here is similar to the way Pc 4 binds to cytosolic organelles (e.g. mitochondria, lysosomes or vacuoles) in mammalian cells. Miller et al., Toxicol Applied Pharmacol 224(3):290-9 (2007).

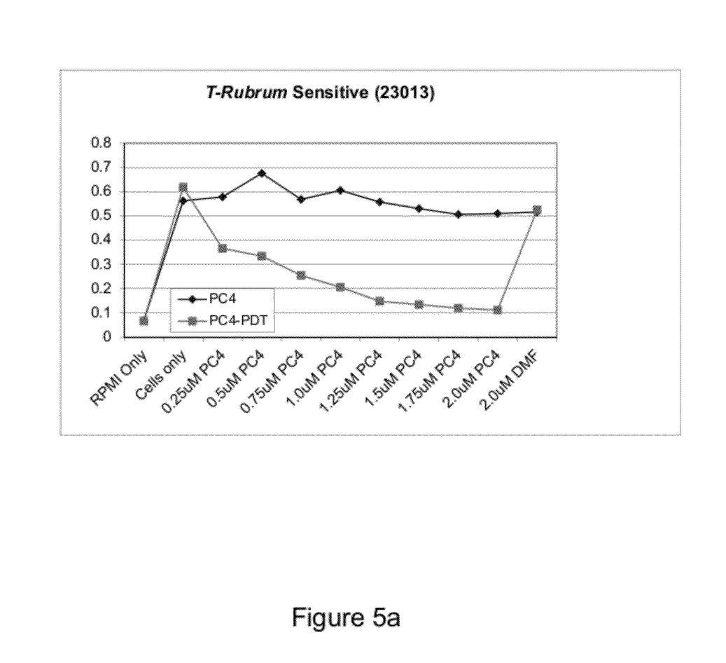

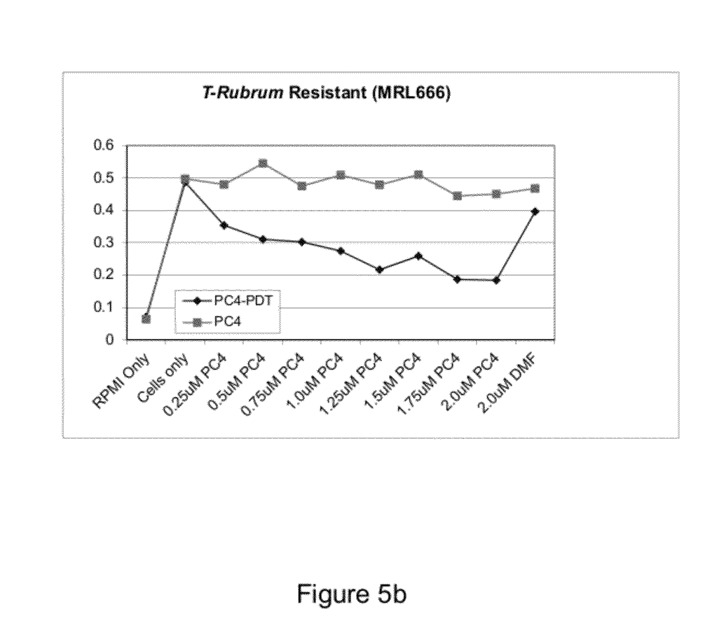

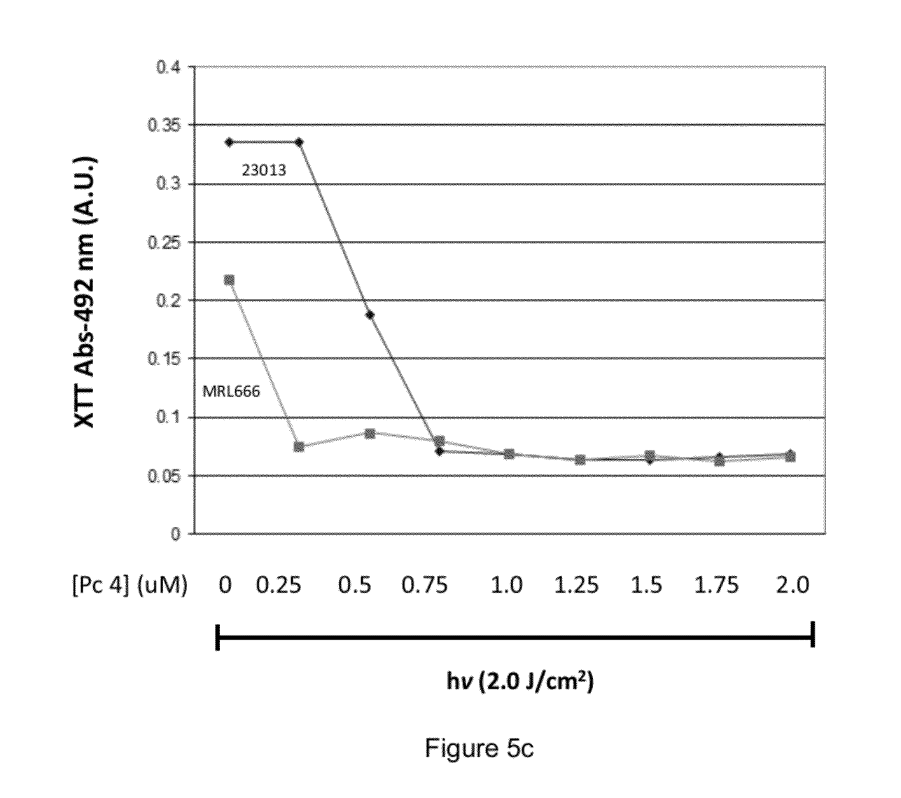

It was found that photoirradiation with 675 nm red light of T. rubrum pre-treated with Pc 4 results in decreased growth and metabolic activity as measured by the Turbidity and XTT assay, respectively. Terbinafine sensitive (23013) and resistant (MRL666) strains of T. rubrum were grown onto Sabouraud potato agar plate for at least 24 hours at 28° C. Microconidia were separated from most of the hyphae form before treating with Pc 4-PDT. Preliminary experiments varying Pc 4 concentration up to 2 μM showed that a 2-hour incubation time was sufficient to establish a growth inhibitory response following irradiation with a 675 nm red light using an LED array (EFOS, Mississauga, Ontario, Canada) using fluence of 2.0 J/cm² (1.0 milliwatt/cm², max 670-675 nm) at room temperature for both 23010 and MRL666 strain as seen in FIGS. 5a and 5b. XTT reduction assay was performed as previously described and shown in FIG. 5c.

### Example 3

### Nail Absorption of Topically Applied Pc 4

Because drug penetration into and through the nail plate is always a challenge in the treatment of onychomycosis, the nail absorption of topically applied Pc 4 was also investigated. Using human nail clippings, it was found that a significant amount of Pc 4 fluorescence can be detected within the nail plate after 1 hour of Pc 4 incubation. Considering that these samples were healthy nails, it appears that diseased/dystrophic nails whose barrier functions have been compromised may even have better Pc 4 absorption. Nonetheless, in the clinical protocol, the inventors specified that Pc 4 will be applied not only on the nail plate but also on the exposed subungual tissue and periungual area. Absorption into the nail matrix through the periungal skin is expected because Pc 4 has long been shown to penetrate through the stratum corneum and epidermis, upon topical application.

### Example 4

### Use of Pc 4 in the Photodynamic Therapy of a Fungal Infection in Humans

A controlled pilot clinical trial involving serial sessions of Pc 4-PDT in adults with culture positive fingernail or toenail onychomycosis will be conducted. Males and females, 18 years and older will be enrolled. Because toenail involvement is more common than fingernail involvement, it is presumed that more toenail onychomycosis cases will be recruited. It is also estimated that there will be a predominance of males in the study due to the natural male preponderance of the disease. More than one nail needs to be involved because at least one other nail will be designated as an untreated control. Patients must be off systemic antifungals for at least 4 weeks, and off topical therapies for at least 2 weeks. Excluded are those with active history of photosensitivity (e.g. xeroderma pigmentosum, lupus erythematosus, porphyria, severe polymorphous light eruption, solar urticaria), any medical condition that could be aggravated or may cause extreme discomfort during the study period. Women of childbearing potential who are pregnant or attempting to become pregnant are excluded from this study. Those women of child-bearing potential who do not wish to become pregnant must agree to utilize a birth control which results in a failure rate of less that 1% per year during the study.

The Pc 4 (NSC 676418) drug supply will be obtained in bulk from the NCI-CTEP. The bulk Pc 4 will then be sent to the Investigational Pharmacy at University Hospitals Case Medical Center (UHCMC). Investigational pharmacists will formulate Pc 4 to 0.1 mg/ml using a vehicle consisting of 70% ethanol and 30% propylene glycol. This packaged formulation will be delivered to the Skin Study Center on the day of treatment. The nail/nails to be treated will be clipped to the shortest length possible. The compounded Pc 4 will be applied by the investigator via a finger cot, over the periungual skin and all exposed areas of the nailbed, as well as over the nail plate. The amount of Pc 4 to be applied will be measured via a pipette, allotting about 5 microliters of the solution per nail unit. The nails will then be fitted/gloved with an opaque material during the incubation time of one hour, to prevent premature photoactivation of Pc 4.

For photoexposure, red light from a novel custom-built portable LED array (Photomedex, Philadelphia PA) will be delivered over the Pc 4-treated area. This 80-LED design emits 675 nm light with an irradiance of 40 mW/cm². An alternative light source is a more conventional stand-up Omnilux unit (Photomedex, Philadelphia Pa.) that has a surface illumination of 15×20 cm, and an irradiance of 95 mW/cm². If this bigger light source is used, the rest of the hands/feet that are unaffected will be covered by opaque cloth so as not to undergo unnecessary red light exposure. The in vitro data on C. albicans and T. rubrum indicated susceptibility at 2 J/cm². Due to properties of human tissue that limit the actual dose of Pc 4 delivered in vivo, the inventors will treat with 50 J/cm²—a dose that has been well-tolerated in prior Phase 1 studies and requires about 15 minutes to deliver. Depending on the number of nails that have the disease in each patient, one or two nails will be designated as untreated controls. No Pc 4 will be applied on these nails, and they will be covered with opaque material during the photoexposure phase.

Local toxicity will be evaluated clinically. The grade of toxicity will be determined by that parameter with the highest level of response under the categories of ulceration and pain. The toxicity data will be tabulated and 95% confidence interval will be estimated. The inventors expect serial Pc 4-PDT to demonstrate safety and tolerability similar to prior Phase 1 clinical trials. However, It is possible that the different nail disease severities may have varying tolerance to the treatment. For example, onychomycosis that is accompanied by significant paronychial inflammation may be more sensitive to local toxic effects of Pc 4-PDT. If this presents as a problem, the analysis will be stratified according to cases without active inflammation versus those with active paronychia.

### Example 5

### Verifying that Pc 4-PDT Results in Mycologic Cures in Patients with Onychomycosis

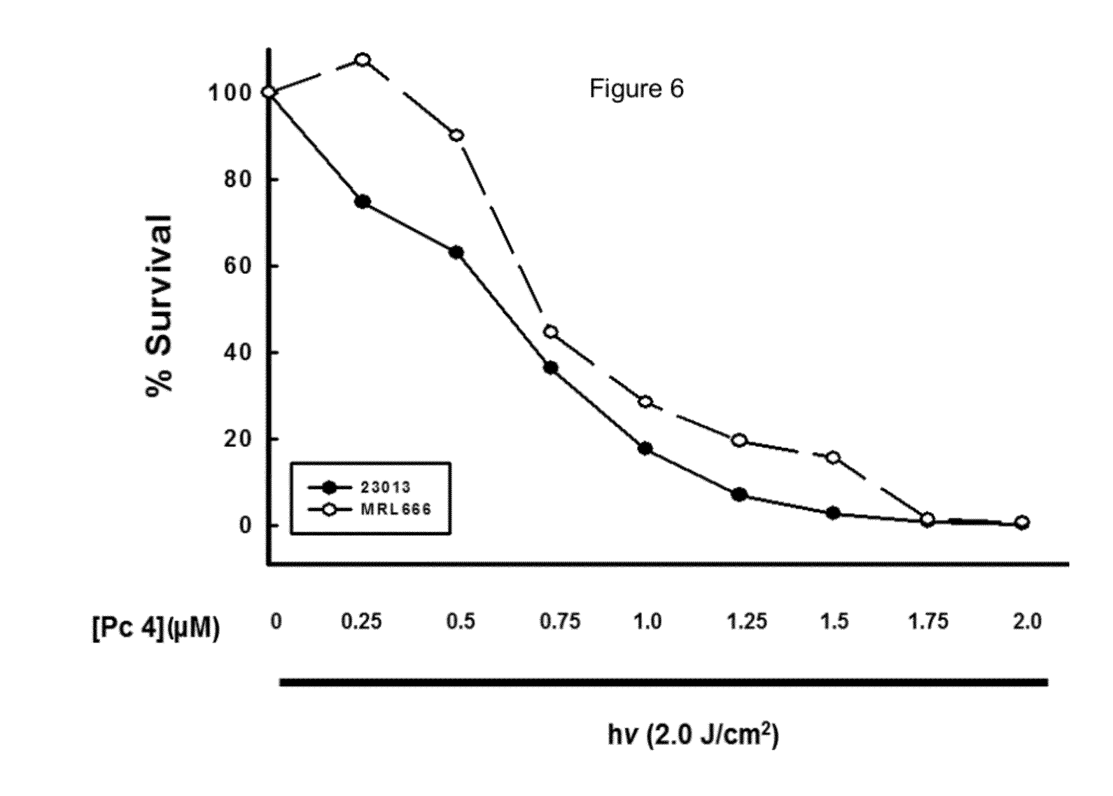

Mycologic cure remains the gold-standard in the evaluation of antifungal therapies and leads to lower recurrence rates. An additional experiment was carried out to demonstrate in vivo what has been observed in vitro, namely, that Pc 4 photodynamic treatment of C. albicans and T. rubrum results in decreased survival as shown by clonogenic assay. Immediately after photoirradiation, treated and control samples were diluted and spread 100 μL onto Sabouraud potato agar plates (Becton Dickinson, New Jersey, USA). All plates were incubated at 28° C. and colonies were counted within 5 days. Pc 4 photodynamic treatment resulted in a dose-dependent decrease in the survival for both terbinafine-sensitive (23013) and terbinafine-resistant (MRL666) strains. See FIG. 6.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of photodynamic treatment of a fungal infection in a subject in need thereof by administering a therapeutically effective amount of a phthalocyanine compound or a pharmaceutically acceptable salt thereof to the subject and activating the phthalocyanine compound with light.

2. The method of claim 1, wherein the phthalocyanine compound has a structure according to formula I: 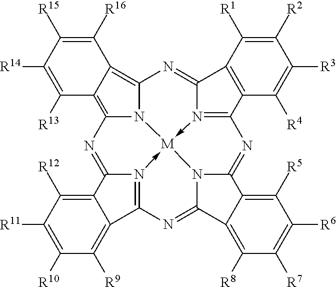
wherein R¹-R¹⁶ are each be independently selected from the group consisting of hydrogen, halogen, nitro, cyano, hydroxy, thiol, amino, carboxy, aryl, heteroaryl, carbocyclyl, heterocyclyl, C₁₋₂₀alkyl, C₁₋₂₀alkenyl, C₁₋₂₀alkynyl, C₁₋₂₀alkoxy, C₁₋₂₀acyl, C₁₋₂₀alkylcarbonyloxy, C₁₋₂₀aralkyl, C₁₋₂₀hetaralkyl, C₁₋₂₀carbocyclylalkyl, C₁₋₂₀heterocyclylalkyl, C₁₋₂₀aminoalkyl, C₁₋₂₀alkylamino, C₁₋₂₀alkylthio, C₁₋₂₀hydroxyalkyl, C₁₋₂₀alkyloxycarbonyl, C₁₋₂₀alkylaminocarbonyl, C₁₋₂₀alkylcarbonylamino, and C_{1-10alkyl}-Z—C₁₋₁₀alkyl; Z is selected from S, NR¹⁷, and O, and R¹⁷ is optionally selected from hydrogen, C₁₋₂₀acyl, C₁₋₂₀alkyl, and C₁₋₂₀aralkyl;
the axial ligand M is (G)ₐY[OSi(CH₃)₂(CH₂)_{b}N_{c}(R′)_{d}(R″)ₑ)_{f}X_{g}]ₚ;
wherein Y is selected from Si, Al, Ga, Ge, Zn, or Sn;
R′ is selected from H, CH₃, C₂H₅, C₄H₉, C₄H₈NH, C₄H₈N, C₄H₈NCH₃, C₄H₈S, C₄H₈O, C₄H₈Se, OC(O)CH₃, OC(O), CS, CO, CSe, OH, C₄H₈N(CH₂)₃CH₃, (CH₂)₂N(CH₃)₂, (CH₂)ₙN((CH₂)ₒ(CH₃))₂, and an alkyl group having from 1 to 12 carbon atoms;
R″ is selected from H, SO₂CH₃, (CH₂)₂N(CH₃)₂, (CH₂)₁₁CH₃, C(S)NHC₆H₁₁O₅, (CH₂)ₙN((CH₂)ₒ(CH₃))₂, and an alkyl group having from 1 to 12 carbon atoms;
X is a counterion selected from the group consisting of chloride, bromide, nitrate, sulfate, tosylate, phosphate, tartrate, maleate, malate, mesylate, inosate, dimethylphosphonate, methylsulfonate, and sulfonate;
G is selected from OH and CH₃; a is 0 or 1; b is an integer from 2 to 12; c is 0 or 1; d is an integer from 0 to 3; e is an integer from 0 to 2; f is 1 or 2; g is 0 or 1; n is an integer from 1 to 12; o is an integer from 1 to 1 1; and p is 1 or 2.

3. The method of claim 2, wherein substituents R¹, R⁴, R⁵, R⁸, R⁹, R¹², R¹³, and R¹⁶ are each independently selected from hydrogen, halogen, nitro, cyano, hydroxy, thiol, amino,and methyl; and R², R³, R⁶, R⁷, R¹⁰, R¹¹, R¹⁴, and R¹⁵ are each independently selected from hydrogen, halogen, nitro, cyano, hydroxy, thiol, amino, carboxy, aryl, heteroaryl, carbocyclyl, heterocyclyl, C₁₋₆alkyl, C₁₋₆alkenyl, C₁₋₆alkynyl, C₁₋₆alkoxy, C₁₋₆acyl, C₁₋₆alkylcarbonyloxy, C₁₋₆carbocyclylalkyl, C₁₋₆aminoalkyl, C₁₋₆alkylamino, C₁₋₆thioalkyl, C₁₋₆alkylthio, C₁₋₆hydroxyalkyl, C₁₋₆alkyloxycarbonyl, C₁₋₆alkylaminocarbonyl, and C₁₋₆alkylcarbonylamino.

4. The method of claim 2, wherein Y is Si.

5. The method of claim 2, wherein R¹-R¹⁶ are H.

6. The method of claim 2, wherein G is OH and N_{c}(R′)_{d}(R″)ₑ of the axial ligand M is N(CH₃)₂.

7. The method of claim 1, wherein the phthalocyanine compound is Pc4.

8. The method of claim 7, wherein the light has a wavelength of from about 660 nm to about 680 nm.

9. The method of claim 1, wherein photodynamic treatment includes administering from about 0.5 to about 2.0 J/cm² of light.

10. The method of claim 1, wherein the fungal infection is caused by yeast of the genus Candida.

11. The method of claim 10, wherein the fungal infection is caused by Candida albicans.

12. The method of claim 1, wherein the fungal infection is caused by Trichophyton rubrum.

13. The method of claim 1, wherein the fungal infection is onychomycosis.

14. The method of claim 13, wherein the fungal infection is Candidal onychomycosis.

15. The method of claim 1, wherein the subject is a human.

16. The method of claim 1, wherein the phthalocyanine compound is administered topically.

17. The method of claim 1, wherein the phthalocyanine compound administered in a pharmaceutical carrier and has a concentration from about 0.05 mg/ml to about 0.1 mg/ml.
